(19) United States
(12) Patent Application Publication
(10) Pub. No.: US 2004/0076543 A1
(43) Pub. Date: Apr. 22, 2004

(54) System and method for decontamination and sterilization of harmful chemical and biological materials

(76) Inventors: Asaf Zeev Sokolowski, Tel Aviv (IL); Dror Niv, Ramat Gan (IL); Amram Fried, Tel-Mond (IL)

Correspondence Address: Edward Langer, c/o Shiboleth, Yisraeli, Roberts, Zisman & Co., 60th Floor, 350 Fifth Avenue, New York, NY, 10118, US

(21) Appl. No.: 10/388,756

(22) Filed: Mar. 17, 2003

Related U.S. Application Data
(60) Provisional application No. 60/364,582, filed on Mar. 18, 2002.

(51) Int. Cl. A61L 2/00; B01J 19/08

(52) U.S. Cl. 422/22; 422/186; 422/186.04

(57) ABSTRACT

A system for decontamination and sterilization of harmful contaminated biological and chemical materials, the system including a plurality of double dielectric barrier discharge reactor cores, wherein each of the reactor cores includes a plurality of parallel, spaced-apart electrodes arranged as a plurality of adjacent triads defining a gap region between opposing electrical poles for the passage of contaminated materials therebetween, and a housing unit provided with an inlet and an outlet for passing contaminated materials through the system. When an electric power supply is connected to the electrodes of the plurality of reactor cores, a high electric field and a plurality of multi-directional electrical micro-discharges are generated in the gap region to produce reactive radicals, so that when contaminated materials are passed through the gap region, contaminants are decomposed by the radicals, while other contaminant molecules are broken down by the high electric field.

### CROSS-REFERENCE TO RELATED APPLICATIONS

The present application is a continuation of prior, U.S. Provisional Patent Application No. 60/364,582 filed Mar. 18, 2002 by the named inventors, Asaf Sokolowski, et. al., and which assumes the protection of the respective date of filing for the inventive concepts and preferred embodiments described in the prior Provisional Patent Application and which are reintroduced hereinbelow.

### FIELD OF THE INVENTION

The present invention relates to corona reactors, and more particularly, to a plasma reactor of the double dielectric barrier discharge type and its use in a non-thermal plasma-based decontamination and sterilization system.

### BACKGROUND OF THE INVENTION

Plasma may be defined as an electrically conducting medium in which there are roughly equal numbers of positively and negatively charged particles, produced when the atoms in a gas become ionized. It is sometimes referred to as the fourth state of matter, distinct from the solid, liquid and gaseous states.

When energy, such as heat, is continuously applied to a solid, it first melts, then it vaporizes and finally electrons are removed from some of the neutral gas atoms and molecules to yield a mixture of positively charged ions and negatively charged electrons, while overall neutral charge is conserved. When a significant portion of the gas has been ionized, its properties will be altered so substantially that little resemblance to solids, liquids and gases remains. A plasma is unique in the way in which it interacts with itself, with electric and magnetic fields and with its environment. A plasma can be thought of as a collection of ions, electrons, neutral atoms and molecules, and photons in which some atoms are being ionized simultaneously with other electrons recombining with ions to form neutral particles, while photons are continuously emitted and absorbed.

Plasma may be produced in a discharge tube, which is a closed insulating vessel containing gas through which an electric current flows when sufficient voltage is applied to its electrodes.

Normally, air consists of neutral molecules of nitrogen, oxygen and other gases, in which electrons are tightly bound to atomic nuclei. On application of an electric field above a threshold level, some of the negatively charged electrons are separated from their host atoms, leaving them with a positive charge. The negatively charged electrons and the positively charged ions are then free to move separately under the influence of the applied electric field. Their movement constitutes an electric current. This ability to conduct electrical current is one of the more important properties of plasma.

Plasma has been widely studied, different technologies have been developed to obtain different types of plasma, and industrial applications have emerged.

The use of plasma as an inducer of chemical reactions and its application for treating biological and chemical pollutants has been widely known for the past couple of decades. The catalyzing performance of plasma depends on its characteristics, which in turn depend on the type of discharge. The discharge itself depends on the shape of electrodes, on the nature of the inter-electrode region, on the voltage and current waveforms used for producing the plasma.

There are at least four known types of plasma production:

1. Electron beam

2. Pulsed corona discharge

3. Surface discharge

4. Volume silent discharge (dielectric barrier corona discharge)

An electrical discharge is the passage of electrical current through a material that does not normally conduct electricity, such as air. On application of a high voltage source, the normally insulating air is transformed into a conductor, a process called electrical breakdown, and sparks, which are a form of electrical discharge, fly.

There are several types of electrical discharges:

1. Corona—a discharge occurring when a highly heterogeneous electric field is applied to a gas. Typically, a very high electric field is present adjacent to a sharp electrode, and a net production of new electron-ion pairs occurs in this vicinity. The corona typically is characterized by a very low current and a very high voltage.

2. Glow discharge—typically has a voltage of several hundred volts, and currents up to 1 Amp. A small electron current is emitted from the cathode by collisions of ions, excited atoms and photons, and then multiplied by successive electron impact ionization collisions in the cathode fall region.

3. Arc discharge—a high current, low voltage discharge, in which electron emission from the cathode is produced by thermionic- and/or field-emission in vacuum.

Gas phase corona reactor (GPCR) technology enables the use of electrical discharges in order to excite electrons to very high energies, while the rest of the gas stays at ambient temperature. The energized electrons collide with gas molecules producing highly reactive radicals, such as [O²⁻[N²⁻], [OH⁻] and the like, which in turn decompose various contaminants.

Volatile organic compounds (VOCs) are an example of common air pollutants released in a number of industrial processes. Emission of VOCs is conventionally controlled by techniques such as thermal oxidation, catalytic oxidation, activated carbon adsorption, bio-filtration, etc. These technologies are generally expensive and have high energy consumption. Growing world concern for environmental protection has promoted testing and evaluation of a number of alternate techniques for abatement of VOCs.

Non-thermal plasma generated by GPCRs has developed as a cost effective and environmentally friendly method for destroying VOCs, which are common air pollutants released in a number of industrial processes. The majority of the electrical energy supplied to the reactor goes to the excitation of energetic electrons rather than into producing ions and heating the ambient gas. This is a more efficient and cost-effective method of decomposing toxic compounds than conventional methods.

Non-thermal plasma is highly effective in promoting oxidation, enhancing molecular dissociation and producing reactive radicals that cause the enhancement of chemical reactions, thereby converting pollutants to harmless by-products.

GPCRs of the dielectric barrier discharge (DBD) type have historically been used to produce industrial quantities of ozone, which have been used in the air and water purification fields. In ozone-based air purification, contaminated fluid (i.e., a gas or a liquid) is brought into contact with ozone (produced by various methods) while in plasma-based air purification the contaminated fluid is driven through a corona reactor and exposed to plasma. Plasma purification has the advantage of being capable of treating extremely difficult compounds such as perfluorocarbons. Plasma purification is also more efficient than ozone purification, providing removal of a significantly greater weight of contaminant per unit energy input.

In addition to treatment of gases and liquids, plasma purification systems may also be used to treat solids and powders, including concealed or enclosed material. The prior art describes various processes for purification against harmful materials contained within packages. These include treatment by heat, chemicals, various forms of radiation, electron beams, microwave, RF plasma and other forms of electrical discharges. All these methods have disadvantages, such as the inability to traverse the packaging material, cumbersomeness, incomplete and uneven purification, inefficiency, lengthy retention time requirements, damage to contents, and toxic by-products.

It is understood that, in general, harmful chemical and biological materials are not moved without some protective enclosure in the first instance. The primary container of such contaminated materials is therefore itself subject to contamination and must be treated together with the contaminated materials themselves. Hereinafter, the term contaminated materials is used interchangeably with any associated container unless specifically mentioned otherwise.

### SUMMARY OF THE INVENTION

It would be desirable to achieve an effective, rapid, cost efficient, uniform, purification process, enabling a high degree of penetration of packaging, and in which no damage to contents or formation of toxic by-products occurs.

Therefore it would be desirable to provide a dielectric barrier system for the efficient purification of fluids, solid objects, and powders against a wide range of chemical or biological contaminants. Operation of this system would render all surfaces effectively “transparent”, therefore enabling decontamination of not only exposed surfaces, but also of internal surfaces and of material contained within an outer packaging.

Accordingly, it is an object of the present invention to overcome the disadvantages of the prior art and provide a double dielectric barrier discharge (DDBD) system for the conversion of harmful chemical or biological matter, either in a contaminated fluid stream, or present in various forms (such as a solid or a powder) on an exposed or concealed surface, into harmless by-products. The system is designed to achieve uniform and effective exposure of contaminants to the plasma produced by the electrodes of a DDBD reactor core.

In dielectric barrier systems, the energy density at a given voltage is inversely proportional to the distance between pairs of electrodes of opposite polarity. There is a significant drop in energy density as spatial separation from a discharge point is increased, such that energy becomes significantly lower even at points a short distance away from a discharge point. In the multi-electrode crisscross array of the present invention, the geometrical placement of the electrodes increases the efficiency of the system via two parameters.

Firstly, the distance between adjacent electrodes as constructed in accordance with the principles of the present invention is less than the diameter of the electrodes in order to ensure that contaminated material passed through the purification system is exposed to sufficiently high energy density at any point between electrodes. Greater separation distance results in an energy level below a critical minimum in the region between electrodes, enabling contaminated material to pass untreated through this area.

Secondly, the separation between adjacent electrodes defines individual discharge volumes between electrodes. With each electrode having opposite polarity, a plurality of electrical micro-discharge paths is formed from each electrode to its adjacent electrode across adjacent reaction volumes, such that the ionized gas formed can flow from one discharge volume to the next in series. The triad geometrical arrangement of electrodes therefore creates a “pinball” flow path forcing the contaminated material and any container enclosing such material into close proximity with the electrode surfaces, which comprise “hot zones” of high energy. This advantageous arrangement of electrodes also increases the residence time of the ionized gas in the purification system without significantly increasing the size of the system.

Therefore, in accordance with a preferred embodiment of the present invention, there is provided a system for decontamination and sterilization of harmful contaminated biological and chemical materials, the system comprising:

a plurality of double dielectric barrier discharge (DDBD) reactor cores, wherein each of the DDBD reactor cores comprises:

a plurality of parallel, spaced-apart electrodes arranged as a plurality of adjacent triads defining a gap region between opposing electrical poles for the passage of contaminated materials there between, and

a housing unit provided with an inlet and an outlet for passing contaminated materials through the system,

wherein when an electric power supply is connected to the electrodes of the plurality of DDBD reactor cores, a high electric field and a plurality of multi-directional electrical micro-discharges are generated in the gap region to produce reactive radicals, such that when contaminated materials are passed through the gap region, contaminants are decomposed by the radicals, while other contaminant molecules are broken down by the high the high electric field itself and its associated micro-discharges.

The system further comprises:

a manipulating means for moving the contaminated biological and chemical materials through the system;

at least one blower unit for drawing air into the system and exhausting air therefrom; and

at least one filter element for filtering air.

A feature of the present invention is the provision of a plurality of double dielectric barrier discharge reactor cores in which the electrical micro-discharges are homogenous and in which exposure time of contaminated material to the electric field, and contact of radicals to the contaminated material, is high.

An advantage of the present invention is that a wide range of chemical and biological contaminants can be treated.

Another advantage of the present invention is that it may be used to treat concealed contaminants contained within a non-conducting container means, such as a cardboard or plastic package, a postal shipping box or envelope, paper wrapping, and the like, as well as to treat the inner and outer surfaces of the container means itself.

A further advantage of the present invention is that a greater weight of contaminant can be removed per unit energy input compared to other known methods.

Yet a further advantage of the present invention is that high temperatures are not required therefore enabling rapid start-up and low maintenance costs and avoiding damage to the contents of the package.

Still another advantage of the present invention is that it is cost-effective and environmentally friendly.

Additional features and advantages of the invention will become apparent from the following drawings and description.

### BRIEF DESCRIPTION OF THE DRAWINGS

For a better understanding of the invention with regard to the embodiments thereof, reference is made to the accompanying drawings (not to scale), in which like numerals designate corresponding sections or elements throughout, and in which:

FIG. 1a is cut-away, general view of a DDBD purification system, constructed and operated in accordance with the principles of the present invention in a preferred embodiment thereof,

FIG. 1b is a general representation of a pair of sub-reactor cores of FIG. 1a;

FIG. 2 is a perspective view of a single electrode of the purification system of FIGS. 1a and 1b;

FIG. 3 is a perspective view of the reactor core of another embodiment of the purification system of the present invention;

FIG. 4 is an enlarged, axial view of the arrangement of a triad of electrodes illustrating the airflow through the gap region between oppositely charged poles, in accordance with the principles of the invention;

FIG. 5 is a perspective view of a system for decontaminating and sterilizing contaminated materials in accordance with a preferred embodiment of the invention;

FIG. 6 is another embodiment of the system of FIG. 5; and

FIG. 7 is yet another embodiment of the system of FIG. 5.

### DETAILED DESCRIPTION OF THE PREFERRED EMBODIMENTS

The present invention relates to a double dielectric barrier discharge (DDBD) system for decontamination of harmful material, such as contaminated powder on the exposed surface of packaging material, such as postal envelopes, or concealed within wrappings. Furthermore, the device and system also is effective in sterilization of packaged objects, such as bandages. This purification process, in a preferred embodiment of the present invention is used for sterilization of biological agents, such as anthrax, and detoxification of harmful chemicals, such as mustard gas, in a range of applications from industrial production lines to mail sorters.

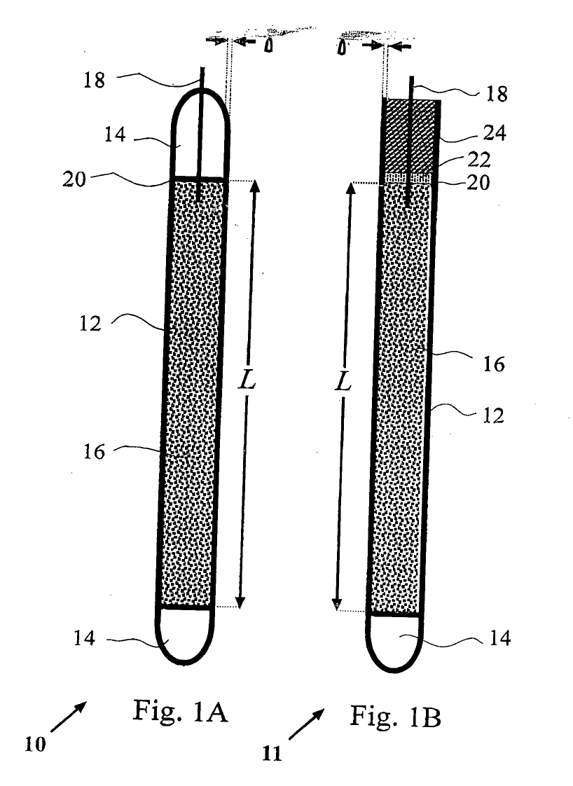

FIG. 1a is a general, sectional view of a DDBD purification system, constructed and operated in accordance with the principles of the present invention in a preferred embodiment thereof.

In a preferred embodiment of the invention, the DDBD reactor core comprises at least one pair of sub-reactor cores which are individually enclosed in modular housing provided with a quick-connect electrical connector to connect the electrodes with a power supply, and with an inlet and outlet for a cooling medium, such as air or oil. The modular housing of pairs of sub-reactor cores lends itself to scaling the decontamination system in accordance with the particular size and quantity of items to be sanitized or decontaminated.

DDBD purification system 10 comprises a plurality of DDBD reactor cores, each comprising a pair of negatively charged and positively charged sub-reactor cores 12a and 12b, respectively. Sub-reactor cores 12a and 12b are modularly designed being individually detachable and removable from housing unit 14. Housing unit 14 is shown, for clarity, in a sectional view without a cover panel. The sub-reactor cores 12a and 12b are fitted through a plurality of openings (not shown) in this cover panel. The openings are blocked by the presence of sub-reactor cores 12a/12b to increase the efficiency of purification during operation of the system 10.

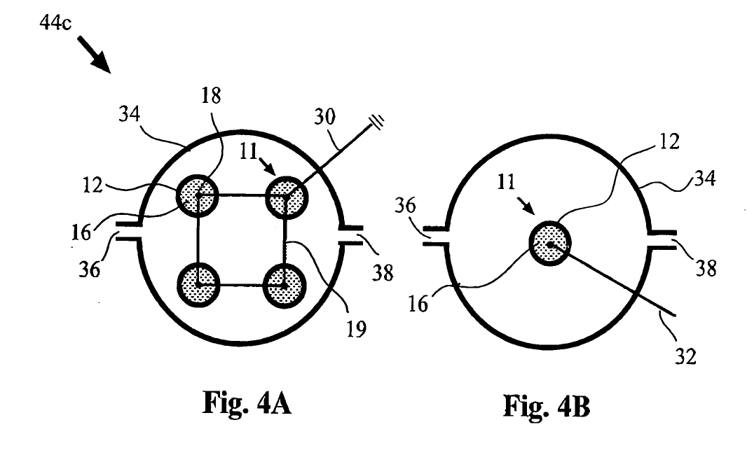

The sub-reactor cores 12a and 12b are arranged opposite each other in a generally parallel orientation forming a series of crisscross triad arrays (as in FIG. 1b and shown in detail in FIG. 4). Sub-reactor cores 12a and 12b have common cross-sectional shapes and equal cross-sectional dimensions. A gap region G is formed between the oppositely charged electrodes in sub-reactor cores 12a/12b which allows passage of contaminated material 16 for processing through the sum of overall micro-discharges along the gap G between the plurality of oppositely charged sub-reactor cores 12a/12b.

When contaminated materials 16 passes into inlet 18 by the movement of a manipulating means 24, such as a conveyer belt as in a preferred embodiment of the invention, contaminated materials 16 moves through gap region G anid is exposed to the fill effects of the micro-discharges produced by electrodes 12 arranged in parallel rows in oppositely charged sub-reactor cores 12a and 12b until contaminated materials 16 exits the system 10 at outlet 20. A dielectric breakdown occurs within the gap region G that creates multi-directional electrical micro-discharges. The electrical micro-discharges depend on the characteristics of the electrodes used, on the nature of the inter-electrode region, and on the voltage and current waveforms used for producing the plasma.

The high electric field excites electrons to very high energies. The energized electrons then collide with background gas molecules producing highly excited ions and radicals, such as [O²⁻], [N²⁻], [OH⁻], inside the purification system 10. These products are directly employed to dissociate and decompose contaminants.

The power supply may be a direct current, or preferably an alternating current power supply The power supply should be capable of producing potential difference between oppositely-charged terminals, preferably, but not necessarily, in the range 10-20 kV and frequency should be preferably, but not necessarily in the range 50-2000 Hz. Furthermore, in a preferred embodiment of the invention, a noble gas, such as Ar or He, is introduced into the system 10 in order to increase energy efficiency of the plurality of sub-reactor cores 12a/12b.

At least one air blower 22a is provided to help circulate and evenly distribute the gas within housing 14. Depending on the volume of air within system 10, a second blower 22b is provided, as seen in the preferred embodiment of the invention in FIG. 1a. At least one filter (not shown) is also provided for filtering the air drawn into system 10, so that the micro-discharges are produced under optimum conditions, and optionally, to filter the exhausted air exiting system 10.

In a preferred embodiment of the system of the invention illustrated in FIG. 1a, contaminated materials 16 is moved through gap G by a manipulation means, such as conveyer belts 24 and a system of rollers 26 connected by conventional mechanical means, such as belts 28, to one or more motors M.

FIG. 1b is a general representation of a pair of sub-reactor cores of FIG. 1a. These sub-reactor cores 12a and 12b comprise physically identical arrangements of electrodes 12 in sealed modular units 35, but their electrodes 12 have oppositely charged poles. The series of electrodes 12 are connected within their respective sub-reactor cores 12a/12b by a conductor element 30 in electrical contact with an externally mounted quick-connection type connector 32, as is known to those skilled in the art. When inserted into position in the housing 14 (see FIG. 1a), connector 32 makes contact with a receptor (not shown) which is connected to the power supply (not shown) for operating the decontamination and sterilization system of the invention. By applying a high alternating voltage, preferably in the range of 10-20 kV, to electrodes 12 through connectors 32, a high electric field is developed across the gap region G.

An inlet 34 and outlet 36 are provided for each sub-reactor 12a/12b for introduction and removal, respectively, of either cooling air or a cooling fluid, such as cooling oil commonly used for cooling electrical components operating at high voltages.

The pair of oppositely charged (+) and (−) sub-reactor cores 12a/12b working together comprise the basic reactor core of the double dielectric barrier discharge system 10 as in FIG. 1a. The electric micro-discharges produced between the oppositely charged electrodes 12 when sub-reactor cores 12a/12b are connected to the power supply (not shown) are dispersed in a multi-directional manner throughout the length of the gap region G which is adjusted to accommodate the passage of various-sized containers possibly concealing contaminated materials 16.

The electrodes 12 are advantageously arranged in adjacent sets of triads, as indicated by the triangle 38 comprising two electrodes 12 from sub-reactor 12b and an oppositely charged electrode 12 from sub-reactor 12a, so as to maximize the strength of the electric field and the density of electrical micro-discharges through which contaminated materials 16 must pass.

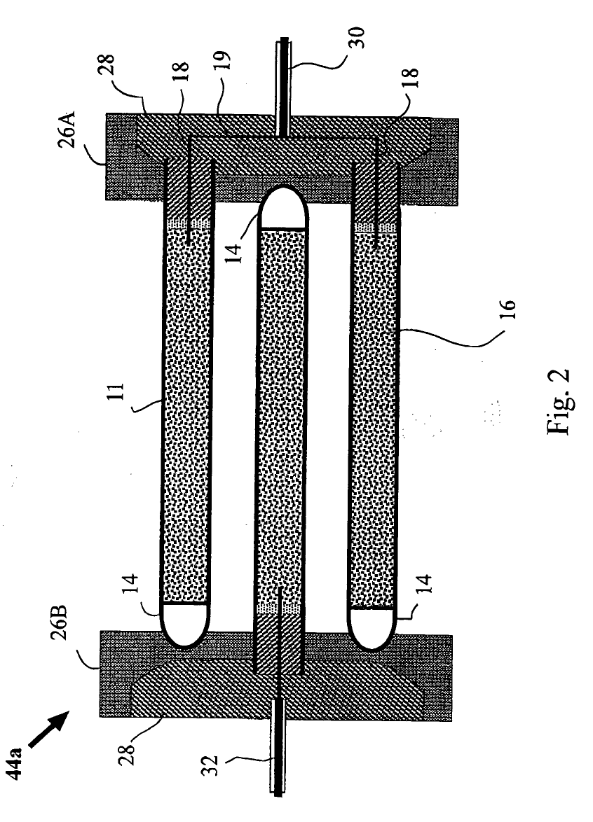

FIG. 2 is a perspective view of a single electrode of the purification system of FIGS. 1a and 1b.

Electrode 12 comprises a hollow tube 40 of conductive material such as silver nitrate AgNO₃, surrounded by a dielectric jacket 42, formed from a material such as ceramic or borosilicate glass, Teflon, and the like, having a high dielectric constant.

Alternatively, the conductive material 40 of electrode 12 may comprise metallic wire, film or powder, carbon wire or film and electricity conducting liquids and gels as is known to those skilled in the art. Note that dielectric jacket 42 extends beyond the ends of tube 40 to prevent unwanted electrical arcing when a voltage is applied to the conductive ends of electrode 12.

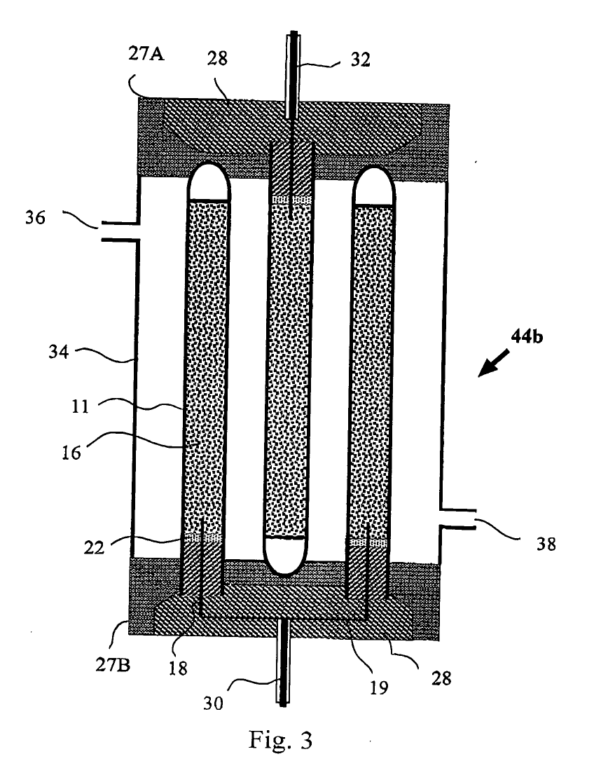

FIG. 3 is a perspective view of the reactor core 50 of another embodiment of the purification system of the present invention.

Referring now to FIG. 3 in detail, a plurality of electrodes 12 are fixed at their proximal and distal ends into frames 44 and 46 respectively in parallel rows. In a preferred embodiment of the invention, frames 44 and 46 are formed from any dielectric material which is not attacked by plasma, has sufficient durability, and is temperature resistant, such as PVC or preferably ceramic.

By applying a high alternating voltage, preferably in the range of 10-20 kV, to series of electrodes 12, connected by conducting wires 48, a high electric field is developed across gap region G between opposite poles of electrodes 12 and a high energy density is developed within reactor core 50.

In a preferred embodiment of the invention, air or cooling oil, such as silicon oil utilized in high voltage transformers, is placed within frame 44 and is passed through the hollow center of the plurality of electrodes 12a/12b in order to enable temperature control of the system. Alternatively, passage of fluid as shown by arrow 56 may be achieved by a pump and heat exchange unit (not shown).

The presence of an insulating fluid such as oil, has the further advantage of preventing oxidation of the electrode surface which may occur as a result of an air gap (not shown) remaining between dielectric material 40 and jacket 42 (see FIG. 2). This is a common problem in the non-thermal plasma field.

An additional advantage of cooling by oil, rather than air, is that it provides a solution to the problem of electrical arcing between exposed anode and cathode potentials by providing an insulating barrier. The electrical properties of oil placed within frame 44 prevent the fatal possibility of arcing which invariably leads to further breakdown of the purification system.

FIG. 4 is an axial view of a triad of electrodes and the direction of airflow through the gap region C between oppositely charged electrodes 12 in accordance with the principles of the invention. The arrangement of adjacent electrodes 12 of opposite charge are shown to form an isosceles triangle 38 (dashed lines) and the direction of airflow (arrows) between the opposite poles of the electrodes 12.are also indicated. Electric micro-discharges (shown by jagged line paths 60) occur between oppositely charged poles when electrodes 12 are connected to an electrical power source (not shown). Note that there are multiple micro-discharge paths 60 and in practice, these are multi-directional as well.

Electrodes 12 are arranged as adjoining reactor cores of three electrodes (triads) set at fixed distances so as to form an isosceles triangle 38 between inversely charged cross-pairs of electrodes 12. The addition of single electrode 12 (anode or cathode, depending on placement) to the base triad electrode group, as in FIG. 4, creates yet another triad, up to any required number of triads. Electrodes 12 are charged so that every two diagonally adjacent electrodes 12 are inversely charged, i.e., every positively charged electrode 12 is in close proximity to a negatively charged electrode 12 and vice versa.

The invention relates to the purification of packages and the articles contained therein, in applications ranging from industrial production lines to mail sorters. This purification process may be used for sterilization of biological agents, such as anthrax and detoxification of harmful chemicals, such as mustard gas.

In this aspect the general structural and electrical relation between electrodes is sustained by adjusting the voltage, while allowing for a suitable gap G for the introduction of the contaminated materials to be purified. The manipulation of the contaminated materials increases their exposure to the plasma environment. Furthermore, the introduction of noble gasses increases energy efficiency within the system of the invention.

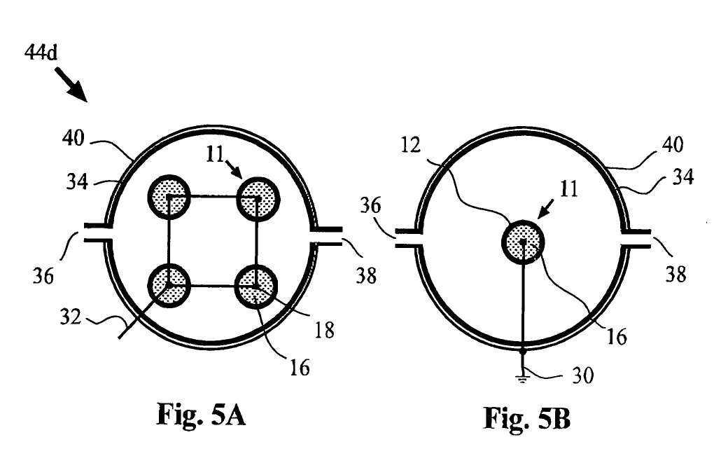

Referring now to FIGS. 5-7 in general, alternative embodiments 70, 80, 90 of the purification system of the present invention are shown. These embodiments are intended for use in purification of material such as powder on the surface of or contained within a container enclosing contaminated materials 16, such as a package or envelope, or the sterilization of packaged objects such as bandages.

The purification systems 70, 80, 90 comprise rows of oppositely charged electrodes 12 of common cross-sectional dimensions, positioned so as to form a criss-cross arrangement of electrodes 12, connected to a high-voltage power supply (not shown). An air gap region G is formed between adjacent electrodes 12. This arrangement is substantially similar to that described with reference to FIG. 1, although the distances between adjacent electrodes 12 may be significantly greater to accommodate the requirements for passage of different sized container means 16.

When contaminated materials 16 (shown as a package) is placed within gap region G, and a high voltage is applied to electrodes 12, a dielectric breakdown occurs which creates electrical micro-discharges along micro-discharge paths 60. The electric field excites electrons to very high energies. The excited electrons then collide with background gas molecules producing highly excited ions and radicals which in turn aid in the dissociation of the chemical contaminants or decomposition of the protective outer coating of biological contaminants, contained within contaminated materials 16 or in or on the surface of the wrappings of contaminated materials 16 Both contaminated materials 16 and its wrapping act as secondary dielectric material between electrodes 12 and therefore do not obstruct the electrical micro-discharges along paths 60 from creating reactive species both within and around contaminated materials 16.

The frequency of the electric voltage producing the micro-discharges along paths 60 (shown by jagged lines) is correlated with the spacing between sets of oppositely charged electrodes 12 in such a manner that achieves an energy density powerful enough for sterilization and/or detoxification.

As mentioned heretofore in relation to FIG. 1, further enhancement of the purification process may be achieved by addition of noble gases such as He or Ar, to the housing 35 of sub-reactor cores 12a/12b in order to ensure even dispersion of energy density.

Due to the crisscross arrangement of electrodes in system 70, electrical micro-discharges occur in a diagonal manner. No purification of contaminated materials 16 will therefore occur in regions 62, located between discharge regions in gap G. In order to achieve full and even purification, it is necessary to manipulate contaminated materials 16 in order to expose all surfaces to regions of electrical micro-discharges 60.

This manipulation may involve various forms of mechanical manipulation, including flipping, sliding, shaking, jerking, dipping, and the like. Mechanical manipulation systems may easily be incorporated into typical conveyance apparatus, including assembly lines, sorting mechanisms, and the like as is known to those skilled in the art.

FIG. 5 is a perspective view of a system for decontaminating and sterilizing contaminated materials in accordance with a preferred embodiment of the invention.

In the example of FIG. 5, mechanical manipulation of contaminated materials 16, shown herein, by way of example, as concealed in a package, is achieved by the plurality of electrodes 12 themselves, which act both as the inducers of non-thermal plasma and as a manipulating means for moving the package of contaminated materials 16.

In this preferred embodiment of the invention, plurality of electrodes 12 is connected to a motorized apparatus (not show) that continuously turns these electrodes 12 around their central axes, effectively creating one system 70 which both conveys and purifies the package of contaminated materials 16 and its contents. Furthermore, the rotation of the plurality of electrodes 12 helps dissipate operating heat and advantageously acts as a cooling mechanism for the electrodes 12.

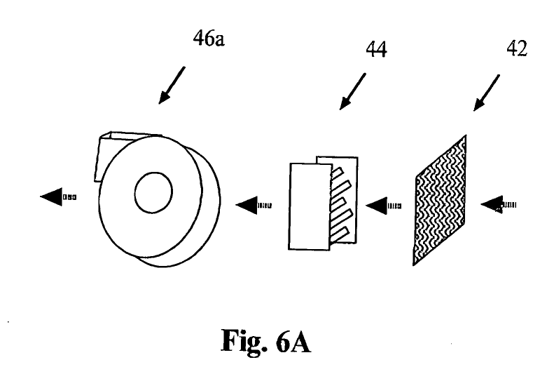

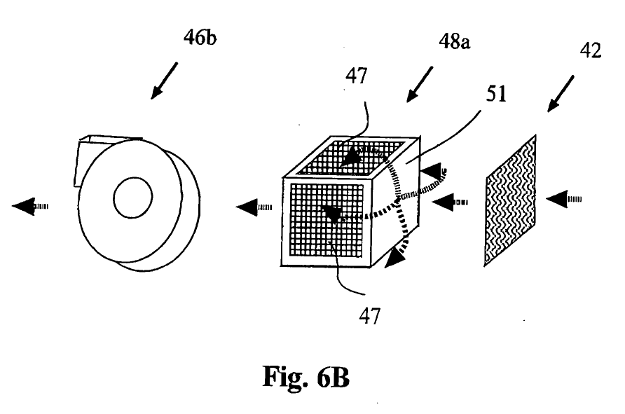

FIG. 6 is another embodiment of the system of FIG. 5.

Note that the purification and decontamination system 80 shown in this embodiment provides for an alternative manipulating means. The plurality of electrodes 12 remain stationary and conveyance of contaminated material 16, such as a package, is achieved by placing rolling cylinders 26 between pairs of adjacent electrodes 12 of the same charge. Rotation of cylinders 26 is achieved by connecting them to a motorized apparatus (not shown), such as a gear mechanism as is known to those skilled in the art.

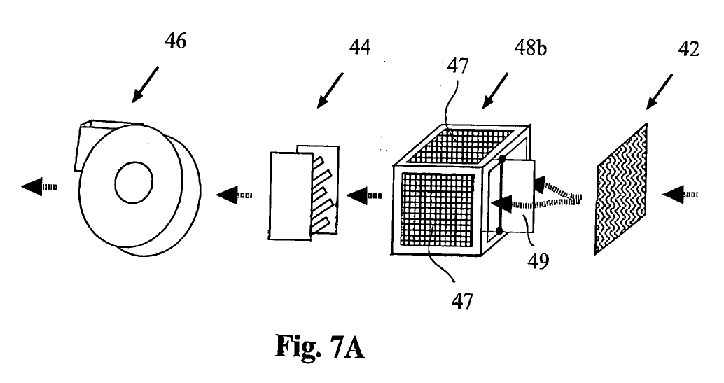

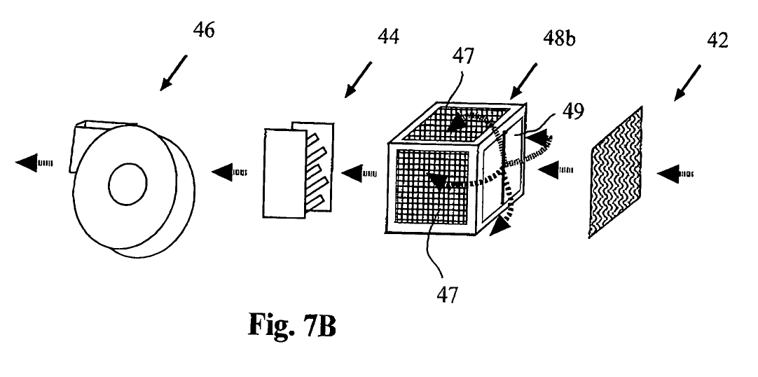

FIG. 7 is yet another embodiment of the system of FIG. 5. A package of contaminated materials 16 is transported by a conveyer belt 24 connected to rolling-cylinders 26 which are connected to a motor means (not shown). In this embodiment 90, a cluster of same polarity electrodes 12 is positioned between each pair of adjacent rolling-cylinders 26 to optimize generation of a high electric field and production of electrical micro-discharges.

The alternative embodiments 70, 80, 90 of the present invention utilize a unique adaptation of a DDBD system to efficiently achieve even sterilization or detoxification of a package and its contents. They require little retention time and neither harm the package content nor create damaging by-products. Because of this, they also have a wide range of applications ranging from use in industrial production lines to use in mail sorters.

Unlike prior art systems, which mainly treat the surface of packages, the purification system of the present invention is able to thoroughly penetrate the interior of a package, ensuring effective purification of the contents and surrounding packaging material.

The present invention operates at ambient temperature, eliminating the need for the relatively high power that is required for systems which operate at elevated temperatures.

The decontaminating device of the present invention therefore provides an efficient and environmentally friendly method for removal of a wide range of contaminants, including those contained within a package whether concealed within or exposed on its outer surface.

The present invention is not limited to treatment of solid or powdered materials, but also has obvious application for processing of contaminated fluids, such as toxic gases or liquids as described in the prior-referenced Provisional Application by the named inventors. In these applications, the manipulation means may be conventional gas or fluid pumps driving the fluid between the oppositely charged poles of dielectric barrier discharge reactors substantially as described herein and illustrated by way of example in the accompanying drawings. For the purpose of fluid applications, the inventive system is, of course, confined within a suitable housing as required for fluid systems and designed for this purpose as is known by those skilled in the art.

Having described the present invention with regard to certain specific embodiments thereof, it is to be understood that the description is not meant as a limitation, since further modifications may now suggest themselves to those skilled in the art, and it is intended to cover such modifications as fall within the scope of the appended claims.

We claim:

1. A system for decontamination and sterilization of harmful contaminated biological and chemical materials, the system comprising:
a plurality of double dielectric barrier discharge (DDBD) reactor cores, wherein each of said DDBD reactor cores comprises:
a plurality of parallel, spaced-apart electrodes arranged as a plurality of adjacent triads defining a gap region between opposing electrical poles for the passage of contaminated materials therebetween, and
a housing unit provided with an inlet and an outlet for passing contaminated materials through said system,
wherein when an electric power supply is connected to said electrodes of said plurality of DDBD reactor cores, a high electric field and a plurality of multi-directional electrical micro-discharges are generated in said gap region to produce reactive radicals, such that when contaminated materials are passed through said gap region, contaminants are decomposed by said radicals, while other contaminant molecules are broken down by said high electric field.

2. The system of claim 1 further comprising:
a manipulating means for moving said contaminated materials through said system;
at least one blower unit for drawing air into said system and exhausting air therefrom; and
at least one filter element for filtering air.

3. The system of claim 1 wherein each of said plurality of electrodes is open-ended and hollow.

4. The system of claim 3 wherein each of said plurality of electrodes is oil-cooled.

5. The system of claim 1 wherein each of said plurality of electrodes comprise an electrical conducting element surrounded by a dielectric jacket.

6. The system of claim 5 wherein said dielectric jacket is glass.

7. The system of claim 1 wherein each of said plurality of electrodes is closed at both ends and provided with a metallic wire extending through one of the closed ends,

8. The system of claim 1 wherein said plurality of electrodes is fixedly and perpendicularly disposed in an arrangement between supporting dielectric frame elements positioned at the distal and proximal ends of said electrodes.

9. The system of claim 1 wherein said triads each comprise three adjacent electrodes arranged in the geometry of an isosceles triangle, a first of said three electrodes disposed on one side of said gap region, and a second and third of said three electrodes disposed on the opposite side of said gap region, wherein the terminals of said second and third electrodes are always inversely-charged in relation to said first of said three electrodes when said three electrodes are connected to said electric power supply.

10. The system of claim 2 wherein said manipulating means comprises rotating electrodes around their central axes to move said contaminated materials through said system.

11. The system of claim 2 wherein said manipulating means comprises rolling-cylinders disposed between at least two adjacent electrodes of the same charge.

12. The system of claim 2 wherein said manipulating means comprises a conveyer belt connected to a plurality of rolling-cylinders.

13. The system of claim 2 wherein said manipulating means provides uniform exposure of said contaminated materials to said high electric field and said plurality of micro-discharges.

14. The system of claim 13 wherein said high electric field and said plurality of micro-discharges disintegrates the chemical contaminants of said contaminated materials.

15. The system of claim 13 wherein said high electric field and said plurality of micro-discharges directly decomposes biological contaminants within said contaminated materials.

16. The system of claim 1 wherein said housing unit is filled with noble gas to promote even dispersion of energy density.

17. The system as claimed in claim 1 wherein said decontamination comprises detoxification of harmful contaminated chemical and biological materials in various physical states both concealed within and exposed on the surface of a container means.

18. The system of claim 17 wherein said detoxification is effected at ambient temperature and without damage to said container means.

19. The system of claim 17 wherein said container means comprises non-conducting packing material.

20. The system of claim 1 wherein each of said DDBD reactor cores comprises a pair of sub-reactor cores, a first part of said pair disposed in parallel and opposite a second part of said pair defining said gap region therebetween.

21. The system of claim 20 wherein said first and said second part of said pair of sub-reactor cores each comprises a row of spaced-apart, like-polarity electrodes fixedly arranged lengthwise in parallel within a housing.

22. The system of claim 20 wherein when said pair of sub-reactor cores is connected to an electric power supply, said first part of said pair of sub-reactor cores is oppositely charged in relation to said second part of said pair of sub-reactor cores across said gap region so as to define said plurality of adjacent triads of opposing electrical poles between said first and said second part of said pair of sub-reactor cores.

23. A method for decontamination and sterilization of harmful contaminated biological and chemical materials, said method comprising:
providing a plurality of double dielectric barrier discharge (DDBD) reactor cores, wherein each of said DDBD reactor cores comprises:
a plurality of parallel, spaced-apart electrodes arranged as a plurality of adjacent triads defining a gap region between opposing electrical poles for the passage of contaminated materials therebetween,
providing a housing unit provided with an inlet and an outlet for passing said contaminated materials through said plurality of reactor cores, and
passing said contaminated materials through said gap region,
such that when said electrodes of said plurality of DDBD reactor cores are connected to an electric power supply, a high electric field and a plurality of multi-directional electrical micro-discharges are generated in said gap region to produce reactive radicals, and contaminants are decomposed by said radicals, while other contaminant molecules are broken down by said high electric field.
